(19) United States
(12) Patent Application Publication
(10) Pub. No.: US 2022/0281938 A1
(43) Pub. Date: Sep. 8, 2022

(54) Biotechnology for curing hypothyroidism

(71) Applicant: Brian Haney, Pittsburgh, PA (US)

(72) Inventor: Brian Haney, Pittsburgh, PA (US)

(21) Appl. No.: 17/194,823

(22) Filed: Mar. 8, 2021

(51) Int. Cl. C07K 14/575; G16H 20/10; G06N 3/02

(52) U.S. Cl. CPC C07K 14/575; G16H 20/10; G06N 3/02

(57) ABSTRACT

A method and apparatus for thyroid hormone control using a synthetic module containing thyroid hormone and an intelligent valve control device. In embodiments, the invention consists of an iterative process with three steps. First, blood is measured to detect thyroid hormone concentration. Second, the measured concentration is compared to a target thyroid hormone concentration. Third, the intelligent valve control device optimizes thyroid hormone delivery, correcting for differences between the measured concentration and the target concentration.

### BACKGROUND TO THE INVENTION

The field of the invention rests at the intersection of two broader fields, artificial intelligence and endocrinology. Endocrinology is a medical branch concerned with endocrine glands and hormones, including the thyroid and thyroid hormones. Artificial intelligence refers to computational processes mirroring the human mind's thoughtful deliberation, decision making, and action-oriented behavior. Specifically, the field of the invention is artificial intelligence for drug delivery to patients with hypothyroidism, the most common thyroid disease in the United States.

Hypothyroidism is an endocrine disease affecting an estimated 9,900,000 people in the United States, 37,070,000 in Europe, and 104,000,000 people globally. Conventional methods for curing the disease fail for various reasons leading to ongoing patient symptoms including weight gain, depression, and anxiety. Further, medical reports suggest a common trend is that more than half of patients with hypothyroidism are undiagnosed and untreated. Moreover, there are several hypothyroidism types. For example, Congenital Hypothyroidism (CH) is defined as thyroid hormone deficiency present at birth. Another example is Hashimoto's Disease, a hypothyroid condition in which the immune system attacks the human thyroid gland preventing adequate Thyroxine (T₄) production.

The thyroid is a bilobal gland located at the base of the neck in front of the windpipe. The thyroid's functionality, essentially synthesizing thyroid hormone, is meticulously modeled through a feedback loop. The pituitary gland and thyroid communicate instructions for controlling hormone production and stabilization. The system works on an iterative loop where cells in the pituitary gland determine the body's normal hormonal range, known as the set point. In other words, the thyroid produces hormones, which are secreted into the blood and then carried to every tissue in the body. As such, the Thyroid produces a hormonal variety governing the human body's metabolism.

Thyroid hormone synthesis is a three-step process. First, the hypothalamus produces Thyroid Releasing Hormone (TRS), stimulating the pituitary gland to release Thyroid Stimulating Hormone (TSH), Thyrotropin, which in turn activates the thyroid gland. Second, the thyroid gland excretes Thyroxine (T₄) to the bloodstream. Third, T₄ converts to Triiodothyronine (T₃) through deiodination in peripheral tissues. This synthesis is critical for metabolic control in the human body.

Thyroxine (T₄) converts to the active Triiodothyronine (T₃) within cells and peripheral tissues by deiodinases. As such, in contrast to Thyroxine (T₄), the Triiodothyronine (T₃) molecule contains three iodine atoms. Triiodothyronine (T₃) is the physiologically active thyroid hormone. It controls myocardium properties, heart rate, and vascular function. In fact, Triiodothyronine (T₃) affects almost every process in the body. Some suggest the thyroid gland produces T₃ directly. Although, thyroid disease is typically not treated with Triiodothyronine (T₃) supplementation. However, speculate a Thyroxine (T₄) and Triiodothyronine (T₃) combination might be better. Molecular structures are important because clinical effects resulting from thyroid hormone imbalance are observable at the cellular level.

As such, Thyroid hormone maintenance is extremely critical for adult metabolic activity, and thyroid hormone abnormalities in adolescence can have catastrophic consequences. Thyroid hormone imbalance can have profound effects on the central nervous system. Interestingly, Thyroid hormone receptors are located throughout the brain, highlighting their importance in central nervous system's development and function. Indeed, Triiodothyronine (T₃) and Thyroxine (T₄) also provide feedback to the brain and anterior pituitary gland to regulate thyroid hormone.

In adults, Hypothyroid symptoms include chronic fatigue, depression, impaired memory, and weight gain. In adolescents, Hypothyroid symptoms include poor growth trajectories, limited mental development, and delayed puberty. Additionally, hypothyroidism may cause problems such as sleep apnea, hypothermia, hypoventilation, neuropsychiatric syndromes, peripheral neuropathy, seizure, cerebellar ataxia, and coma.

Thyroid hormone replacement therapy is a chronic and lifetime endeavor for treatment. Thyroid hormone dosage must be established for each patient Individually. Usually, the initial dose is small, with amounts increasing gradually until clinical evaluation and laboratory tests indicate optimal response. The dose required to maintain this response is then continued. It is vital to patients physical and mental health that thyroid hormone treatment have the correct dosage. Under-treatment with thyroid hormone can have deleterious and disastrous consequences including neurochemical imbalances, depression, and fatigue.

Prior to the disclosed invention, the art reflected the idea that hypothyroidism could not be cured. Rather, since the year 1970s, the conventional wisdom has been that hypothyroidism is only treatable by replacing thyroid hormone deficiencies with orally administered levothyroxine (LT₄). And orally administered levothyroxine (LT₄) is still the standard treatment for hypothyroid patients despite its primitive conception five decades ago.

Now, artificial intelligence technologies changing human health. For example, deep learning algorithms enable computer systems to have predictive power far superior to humans in various domains. Deep learning is a process by which machines learn using neural networks. Moreover, reinforcement learning algorithms provide an artificial intelligence structure for goal-oriented behavior. Typically, reinforcement learning programs have three parts. First the model defines an agent-environment relation. Second, a policy defines a decision-making process for the agent. Third, the reward defines the agent's goal, which is optimized according to the policy.

Despite advancements in other health disciplines, artificial intelligence has yet to have an impact for treating hypothyroidism. There are three major problems with daily oral levothyroxine for treating hypothyroidism: (1) Fluctuation in ingestion times and surrounding dietary conditions causes metabolic imbalance, (2) Fluctuation in patient needs causes metabolic imbalance, and (3) Fluctuation in brand name may cause metabolic imbalance. Thurs, there exists a need for new medical methods for patients to with hypothyroidism. The disclosed invention solves these three problems, providing methods for curing hypothyroidism with automatic monitoring and corrective release to maintain metabolic homeostasis.

### SUMMARY OF THE INVENTION

The invention is a process and system for curing hypothyroidism using artificial intelligence to measure blood and command hormonal secretion through a valve delivery mechanism. First, blood sensors measure hormonal concentration in the blood, storing measurements in memory for further processing the measurement. The sensors measure blood to detect and commit hormone levels of memory, including: Thyroxine (T₄), Triiodothyronine (T₃), Free Thyroxine (FT₄), and other thyroid hormones. Second, the measurements are processed and compared to target hormonal levels, and subsequently produce an automatic command for a valve control and drug delivery mechanism. Third, the valve control subsystem motivates thyroid hormone delivery from a supply of reserved hormone in a module to the patient's bloodstream optimizing hormonal homeostasis.

### BRIEF DESCRIPTION OF DRAWINGS

FIG. 1 illustrates embodiments of the invention where a patient with hypothyroidism (100), uses a synthetic thyroid (101), to supplement thyroid hormonal deficiencies (102), effectively curing the patient for as long as the synthetic thyroid is able to provide the patient's needed hormone.

FIG. 2 illustrates methods for human metabolic control as an information flow model, where a data sensor (200), measures blood for hormonal concentration, and transfers a signal (201), to a processor with memory storage wherein the hormonal measurement concentration is stored and processed (202), to subsequently deliver an open command (205), which is linked to a control valve (206) via a computational coupling (203), triggering thyroid hormone delivery (207), from a thyroid hormone store chamber containing thyroid hormone (204), to the bloodstream (208).

FIG. 3 illustrates embodiments of the invention where a synthetic thyroid draws a blood sample (300), which is collected (301), using a measurement device (302), passing (303), the measurement to a computer processor (304), using a convolutional neural network and reinforcement learning algorithm (305), to command drug delivery from a Thyroxine (T₄) supply (306), which is loaded (307), to a release chamber (308), and delivered (309), to the bloodstream as a liquid drop (310).

### DETAILED DESCRIPTION OF THE INVENTION

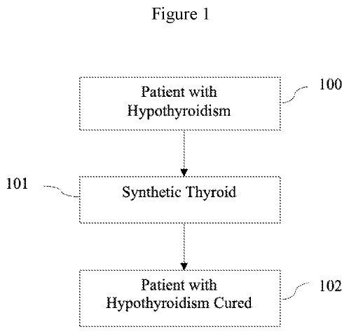

FIG. 1 illustrates embodiments of the invention where a patient with hypothyroidism (100), uses a synthetic thyroid (101), to supplement thyroid hormonal deficiencies (102), effectively curing the patient for as long as the synthetic thyroid is able to provide the patient's needed hormone.

In certain embodiments, the invention is a process by which a patient with hypothyroidism uses a synthetic thyroid to supplement thyroid hormonal deficiencies, effectively curing the patient for as long as the synthetic thyroid is able to provide the patient's needed hormone. The disclosed invention provides a sustainable cure to hypothyroidism. In turn, the invention allows patients suffering from hypothyroidism to live without stresses associated medication management including daily reminders, neurochemical imbalances, and prescriptions. Thus, in certain patient's hypothyroid symptoms are effectively cured without the need for further treatment.

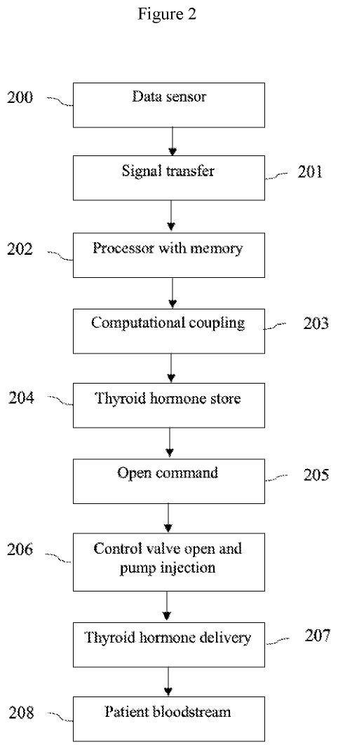

FIG. 2 illustrates methods for human metabolic control as an information flow model, where a data sensor (200), measures blood for hormonal concentration, and transfers a signal (201), to a processor with memory storage wherein the hormonal measurement concentration is stored and processed (202), to subsequently deliver an open command (205), which is linked to a control valve (206) via a computational coupling (203), triggering thyroid hormone delivery (207), from a thyroid hormone store chamber containing thyroid hormone (204), to the bloodstream (208).

In certain embodiments, the invention is a process where a synthetic thyroid draws a blood sample, which is collected, using a measurement device, passing the measurement to a computer processor using a convolutional neural network and reinforcement learning algorithm to command drug delivery from a Thyroxine (T₄) supply, which is loaded, to a release chamber and delivered to the bloodstream as a liquid drop. The delivery stabilizes hormonal homeostasis.

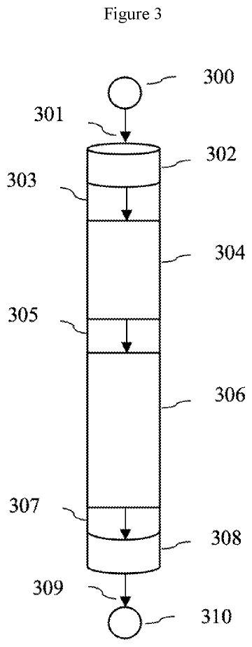

FIG. 3 illustrates embodiments of the invention where a synthetic thyroid draws a blood sample (300), which is collected (301), using a measurement device (302), passing (303), the measurement to a computer processor (304), using a convolutional neural network and reinforcement learning algorithm (305), to command drug delivery from a Thyroxine (T₄) supply (306), which is loaded (307), to a release chamber (308), and delivered (309), to the bloodstream as a liquid drop (310).

In certain embodiments, the present invention is a system for optimizing the hormone level of a patient wherein a data sensor measures blood for hormonal concentration, and transfers a signal, to a processor with memory storage. Then, the hormonal measurement concentration is stored and processed, to subsequently deliver an open command, which is linked to a control valve via a computational coupling. This triggers thyroid hormone release, from a thyroid hormone store chamber containing thyroid hormone, to the bloodstream, by opening the control valve and subsequently commanding the control valve closed.

In certain embodiments, the synthetic thyroid uses a deep learning algorithm to control drug delivery according to commands from a convolutional neural network. The information collected by blood measurements relating to thyroid hormone in the bloodstream may be processed with a convolutional neural network to predict the needed dosing release. According to the prediction, the drug delivery system delivers a specified amount of purified Thyroxine (T₄) to the blood. In turn, the prediction promotes the stabilizing of hormonal homeostasis for the patient over time by effectively linking the measurement and delivery mechanism in the synthetic thyroid via a single neural network.

In certain embodiments, the drug delivery methods use a proximal policy optimization (PPO) algorithm, which is a type of reinforcement learning algorithm, to optimize drug delivery and homeostasis. In general, PPO works by computing an estimator of the policy gradient and iterating with a gradient optimization algorithm. In other words, the algorithm continuously updates the agent's policy based on the old policy's performance. The PPO update algorithm may be defined:

$${θ}_{k + 1} = arg {max}_{θ} {𝔼}_{s , a ∼ {π}_{{θ}_{k}}} [ L ( s , a , {θ}_{k} , θ ) ] . \qquad ( 1 )$$

In equation (1), L(s, α, θₖ, θ) is the objective function, θ are the policy parameters, θₖ are the policy parameters for k experiment. Generally, the PPO update is a method of incremental improvement. Essentially, the algorithm takes multiple steps via gradient descent to maximize the objective.

In certain embodiments, the PPO algorithm's key to the success in controlling drug delivery is obtaining good estimates of an advantage function. The advantage function describes the advantage of a particular policy relative to another policy. For example, if the advantage for the state-action pair is positive, the objective reduces to:

$$L ( s , a , {θ}_{k} , θ ) = min ( \frac{{π}_{θ} ( a \vert s )}{{π}_{{θ}_{k}} ( a \vert s )} , ( 1 + ϵ ) ) {A}^{{π}_{{θ}_{k}}} ( s , a ) . \qquad ( 2 )$$

In equation (2),

$${A}^{{π}_{{θ}_{k}}}$$

is the advantage estimate tor the policy given parameters π_{θ}(α|s), and the hyperparameter ∈ corresponds to how far away the new policy can step from the old while still profiting the objective. Where the advantage is positive the objective increases and the min function puts a limit to how much the objective can increase.

In certain embodiments, the proximal policy optimization algorithm is trained in a simulation environment to develop a reinforcement learning agent. Then, the reinforcement learning agent is embedded to a processor inside the synthetic thyroid. In turn, the reinforcement learning agent sends commands to the drug delivery system according to an optimal decision-making algorithm, which promotes delivery so as to maintain metabolic homeostasis in the patient.

In certain embodiments, an artificial intelligence algorithm processes information relating to the patient's current thyroid hormone level. Then, the artificial intelligence program makes a prediction regarding the patient's needed thyroid hormone for maintaining homeostasis. Next, the prediction informs the amount of thyroid hormone pumped to a drug delivery chamber. Finally, the needed supply necessary for maintaining homeostasis is delivered to the patient's bloodstream.

In certain embodiments, the synthetic thyroid is constructed with nanotechnologies including nanosensors, microprocessors, and carbon nanotubes. The synthetic thyroid may be constructed with multiple carbon nanotubes for performing two essential functions. The first function is measuring the blood for concentrations of Thyroxine (T₄) and Triiodothyronine (T₃). The second function is using the measured information to delivery Thyroxine (T₄) to the bloodstream. Once delivered, the blood will perform deiodinases generating more Triiodothyronine (T₃). The various functions may be performed using commands from a processor within the synthetic thyroid, communicating with nanotubes via nanowiring.

In certain embodiments, the invention is a process and system for curing hypothyroidism using artificial intelligence. First, blood sensors measure thyroid hormone concentration in the blood of the patient. The sensors detect and commit hormone levels of memory, including: Thyroxine (T₄), Triiodothyronine (T₃), Free Thyroxine (FT₄), and other thyroid hormones. Second, the measurements are processed and compared to target hormonal levels, subsequently producing an automatic command for a valve control and delivery mechanism. The commands control the valve control and delivery mechanism such that the open command delivers thyroid hormone to the patient's bloodstream and the close command terminates the delivery process. Third, the valve control and drug delivery system command purified Thyroxine (T₄) delivery from a supply of reserved hormone in a module to the patient's bloodstream optimizing hormonal homeostasis.

It is to be understood that while certain embodiments and examples of the invention are illustrated herein, the invention is not limited to the specific embodiments or forms described and set forth herein. It will be apparent to those skilled in the art that various changes and substitutions may be made without departing from the scope or spirit of the invention and the invention is not considered to be limited to what is shown and described in the specification and the embodiments and examples that are set forth therein. Moreover, several details describing structures and processes that are well-known to those skilled in the art and often associated with biotechnologies are not set forth in the following description to better focus on the various embodiments and novel features of the disclosure of the present invention. One skilled in the art would readily appreciate that such structures and processes are at least inherently in the invention and in the specific embodiments and examples set forth herein.

One skilled in the art will readily appreciate that the present invention is well adapted to carry out the objectives and obtain the ends and advantages mentioned herein as well as those that are inherent in the invention and in the specific embodiments and examples set forth herein. The embodiments, examples, methods, and compositions described or set forth herein are representative of certain preferred embodiments and are intended to be exemplary and not limitations on the scope of the invention. Those skilled in the art will understand that changes to the embodiments, examples, methods and uses set forth herein may be made that will still be encompassed within the scope and spirit of the invention. Indeed, various embodiments and modifications of the described compositions and methods herein which are obvious to those skilled in the art, are intended to be within the scope of the invention disclosed herein. Moreover, although the embodiments of the present invention are described in reference to use in connection with artificial intelligence, ones of ordinary skill in the art will understand that the principles of the present inventions could be applied to other types of biotechnology for a wide variety of applications.

I claim:

1. A method for human metabolic control, the method comprising a synthetic module containing one valve delivery system, the valve opening and closing according to thyroid hormone concentration in the bloodstream, a reinforcement learning agent controlling the valve with an embedded intelligence for optimal thyroid hormone dosing delivery, and one measurement device collecting and processing data, optimizing thyroid hormone dosing delivery according to defined parameters.

2. The method of claim 1, wherein the reinforcement learning agent is a trained proximal policy optimization algorithm, using a trained neural network to ensure the patient's bloodstream maintains homeostasis in thyroid hormone, delivering thyroid hormone to the patient according to a defined set point.

3. The method of claim 1, wherein the defined parameters include a set point, defining the optimal concentration for thyroid hormone in the bloodstream, wherein the difference between a measured concentration is compared to the set point concentration, and an artificial intelligence program manipulates the drug delivery system, minimizing the difference between the measured concentration and set point concentration in the blood.

4. The method of claim 1, wherein the thyroid hormone is purified Thyroxine (T₄).

5. The method of claim 1, wherein the thyroid hormone is a composition containing Thyroxine (T₄) and Triiodothyronine (T₃).

6. The method of claim 1, wherein the thyroid hormone is a composition containing Thyroxine (T₄), Triiodothyronine (T₃), and Free Thyroxine (FT₄).

7. The method of claim 1, wherein the reinforcement learning agent is a proximal policy optimization algorithm, using a deep gradient optimizer, further comprising one convolutional neural network, iteratively improving the difference between a measured concentration of thyroid hormone and set point concentration of thyroid hormone.

8. The method of claim 1, where in the valve is controlled with classical computing system, optimizing drug delivery according to logical principles, performing computations without reference to the current thyroid hormone in the bloodstream.

9. A method for curing hypothyroidism, the method comprising a synthetic thyroid, measuring thyroid hormone in the blood, using an artificial intelligence program, predicting needed delivery dosage, delivering thyroid hormone, according to the dosage defined by the artificial intelligence program, maintaining metabolic homeostasis, controlling thyroid human in the bloodstream.

10. The method of claim 9, wherein the artificial intelligence program is a convolutional neural network, further comprising an input layer, two hidden layers, and output layer, predicting needed dosage.

11. The method of claim 9, wherein the artificial intelligence program is an embedded intelligence, calculating the needed dosage using statistical analysis, using intelligence from a human expert.

12. The method of claim 9, wherein the thyroid hormone is purified Thyroxine (T₄).

13. The method of claim 9, wherein the thyroid hormone is a composition containing Thyroxine (T₄) and Triiodothyronine (T₃).

14. The method of claim 9, wherein the thyroid hormone is a composition containing Thyroxine (T₄), Triiodothyronine (T₃), and Free Thyroxine (FT₄).

15. The method of claim 9, wherein the synthetic thyroid further compromises a carbon nanotube, concealing a thyroid hormone supply, delivering to the blood through a timed-release drug delivery system, according to commands from a micro computing chip, signaling delivery time according to programmed commands.

16. An apparatus for intelligent thyroid hormone delivery, the apparatus comprising a cylindrical tube, further comprising a thyroid hormone supply, a mechanism for measuring thyroid hormone concentration in blood, a device for delivering the hormone supply to the bloodstream, artificial intelligence program which is embedded on a microprocessor in the cylindrical tube.

17. The apparatus of claim 16, wherein the cylindrical tube comprises a carbon nanotube, with a lattice support structure.

18. The apparatus of claim 16, wherein the thyroid hormone supply is purified Thyroxine (T₄).

19. The apparatus of claim 16, wherein the thyroid hormone is a composition containing Thyroxine (T₄) and Triiodothyronine (T₃).

20. The apparatus of claim 16, wherein the thyroid hormone is a composition containing Thyroxine (T₄), Triiodothyronine (T₃), and Free Thyroxine (FT₄).
